(19) United States
(12) Patent Application Publication
(10) Pub. No.: US 2026/0110670 A1
(43) Pub. Date: Apr. 23, 2026

(54) MULTI TECHNOLOGY GAS DETECTION DEVICE AND METHODS FOR ENHANCED ACCURACY, RELIABILITY, AND SAFETY

(71) Applicant: Maarten P.J. Van Laere, Leuven (BE)

(72) Inventor: Maarten P.J. Van Laere, Leuven (BE)

(21) Appl. No.: 19/363,902

(22) Filed: Oct. 21, 2025

Related U.S. Application Data
(60) Provisional application No. 63/709,838, filed on Oct. 21, 2024.

(51) Int. Cl. G01N 33/00; G01K 3/00

(52) U.S. Cl. CPC G01N 33/0006; G01K 3/005; G01N 33/0031

(57) ABSTRACT

Multi-technology gas detection device with automatic calibration, drift compensation, cross validation, adaptive sensing, and cross sensitivity mitigation. A detector integrates at least two different sensing technologies aimed at the same target gas or gases. A control unit compares outputs to (i) automatically adjust baseline or calibration parameters of a traditional component using a calibration stable component, (ii) compensate slow baseline and/or sensitivity changes in real time or periodically using one or more other components, (iii) validate detections via agreement criteria across technologies, (iv) select/switch/weight components based on environmental conditions, and (v) reduce false positives by differential analysis of components with different cross sensitivities. Embodiments cover methane and carbon monoxide detection; independent operation with failover; environment sensors; and packaging on one or more PCBs within one or more enclosures. The device is parameterizable for use in systems subject to a designated safety standard while remaining vendor and recognition status agnostic.

### SEQUENCE LISTING OR PROGRAM

Not Applicable

### TECHNICAL FIELD OF THE PRESENT INVENTION

The invention relates to gas detection systems and devices, and more particularly to detectors that combine multiple, different gas sensing technologies targeting the same gas to improve accuracy, reliability, safety, and operational efficiency.

### BACKGROUND OF THE PRESENT INVENTION

Gas detection is critical in industrial safety, environmental monitoring, and building/fire safety systems. In compliance driven deployments (e.g., systems subject to a designated safety standard such as, but not limited to, UL 2075 or approvals like FM), detectors often must employ recognized/listed components. Many such components drift from baseline and require periodic recalibration and/or replacement, imposing OPEX and CAPEX burdens. By contrast, newer “calibration free” technologies can exhibit long term stability but may lack listings/recognitions required for use in certain safety systems, creating a gap the present invention addresses.

### SUMMARY OF THE INVENTION

The invention provides a multi-technological gas detector that integrates at least two distinct sensing technologies aimed at one or more of the same target gases. A control unit comprising one or more processors and associated memory ingests signals from the different gas sensing technologies to execute some, all, or more of the following independent, complementary aspects:

Automatic calibration of a traditional gas detection component using one or more calibration stable components to reduce or eliminate manual recalibration;

Automatic drift compensation of a traditional gas detection component using one or more other components to confirm or rectify drifting so that slow baseline and/or sensitivity changes are compensated in real time or on a periodic basis;

Cross validation that confirms events based on agreement across two or more different technologies to reduce false positives and negatives;

Adaptive sensing that selects/switches/weights components based on environmental or performance conditions for optimal operation;

Cross sensitivity mitigation via differential analysis between components with different interference profiles to suppress spurious alarms.

These aspects can be implemented independently or in any combination and may be parameterized for systems subject to a designated safety standard without limiting the invention to any particular certification regime. Embodiments include methane and carbon monoxide detectors, independent operation/failover, environment sensors (e.g., temperature/humidity/pressure), algorithmic filtering, and packaging on one or more PCBs within one or more enclosures. The system is parametrically configurable to comply with requirements of a designated safety standard without limiting claims to any single standard or vendor.

In certain embodiments, a compliance critical signaling path is derived exclusively from a gas sensing component designated as compliance critical under a designated safety standard, while one or more additional gas sensing components—whether or not presently recognized under that standard—provide calibration, drift compensation, and corroboration data and cannot supplant the compliance critical signaling path.

### BRIEF DESCRIPTION OF THE DRAWINGS

The accompanying drawings, which are incorporated herein and form a part of the specification, illustrate the present invention and, together with the description, further serve to explain the principles of the present invention and to enable a person skilled in the pertinent art to make and use the present invention.

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a top planar picture of a flammable gas detector.

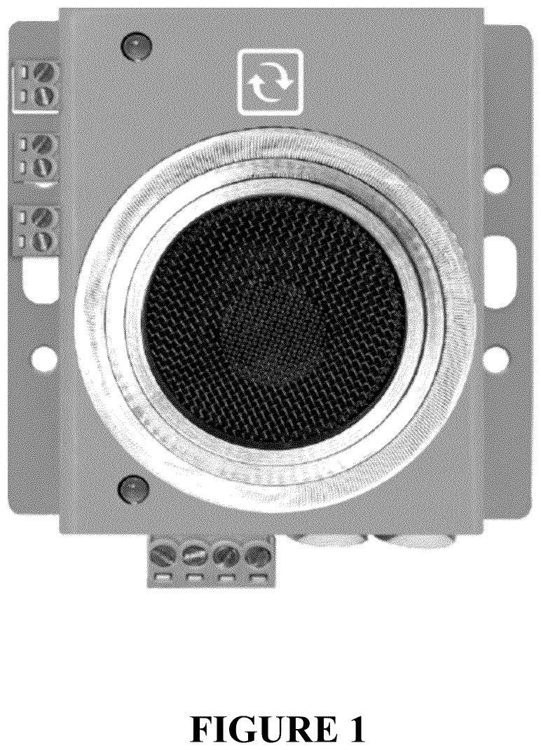

FIG. 2 is a top planar line drawing of the detector.

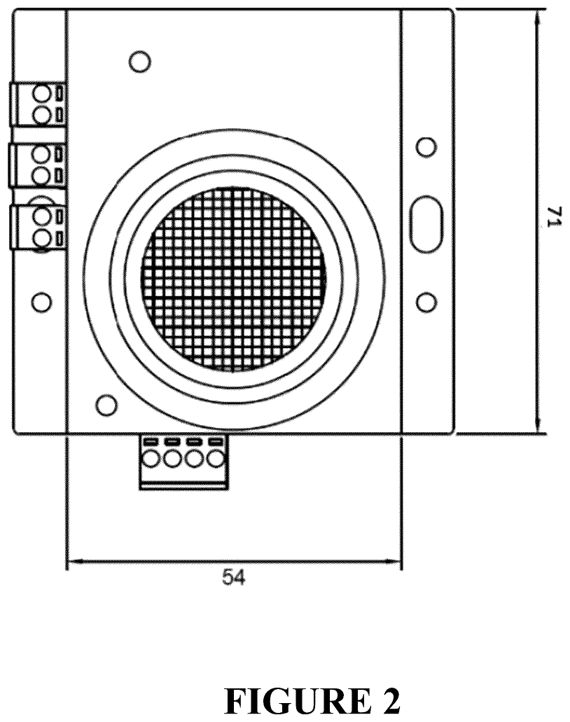

FIG. 3 is a rear planar line drawing of the detector.

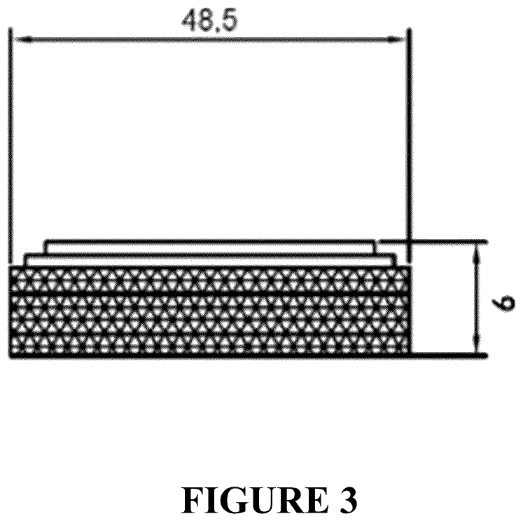

FIG. 4 is a front planar line drawing of the detector.

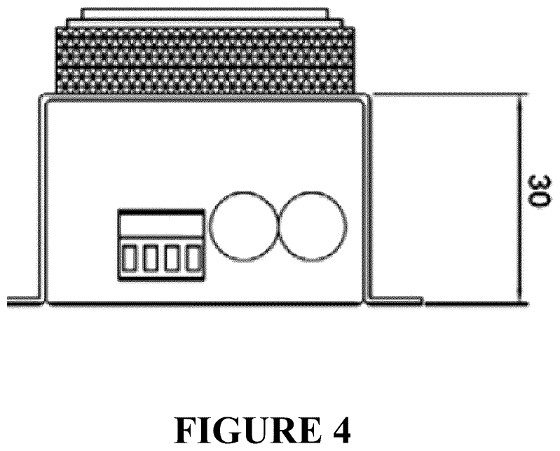

FIG. 5 is a top planar line drawing of a sensor subassembly located at the top of the detector.

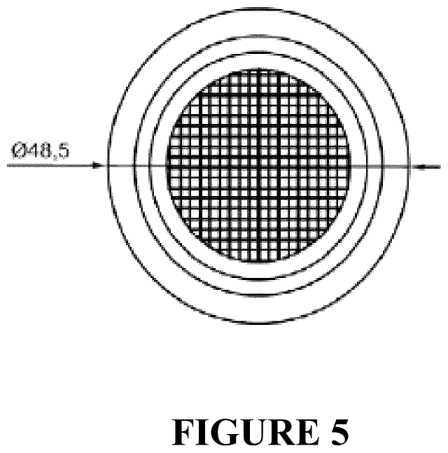

FIG. 6 charts representative sensor metrics.

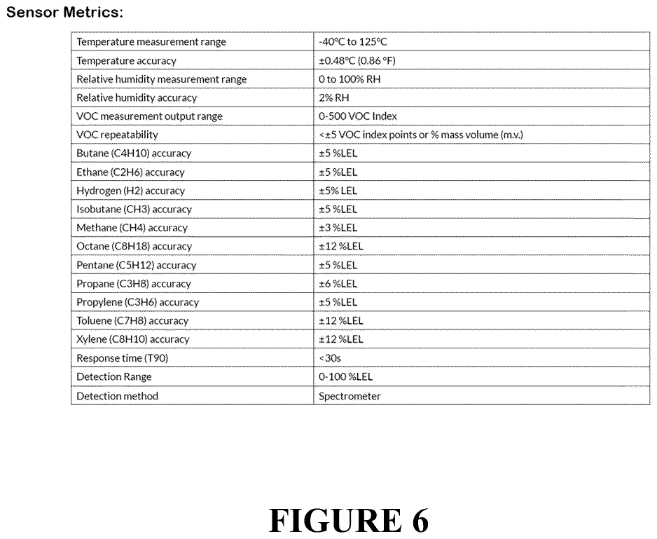

FIG. 7 charts technical specifications.

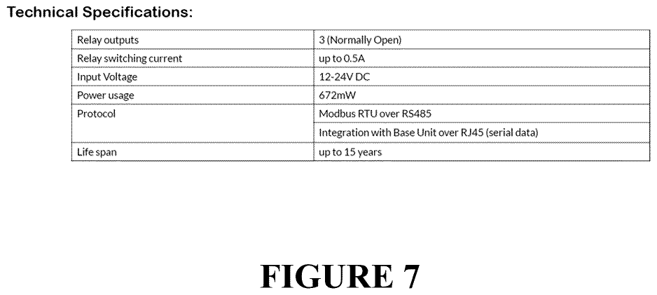

FIG. 8 charts environmental specifications.

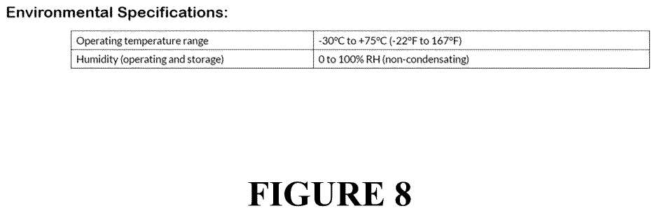

FIG. 9 charts physical specifications.

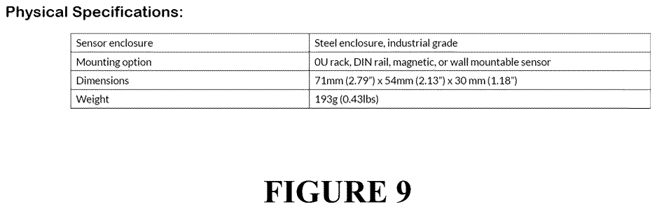

FIG. 10 is a top planar view of the inside of a flammable gas detector with the different gas sensing components.

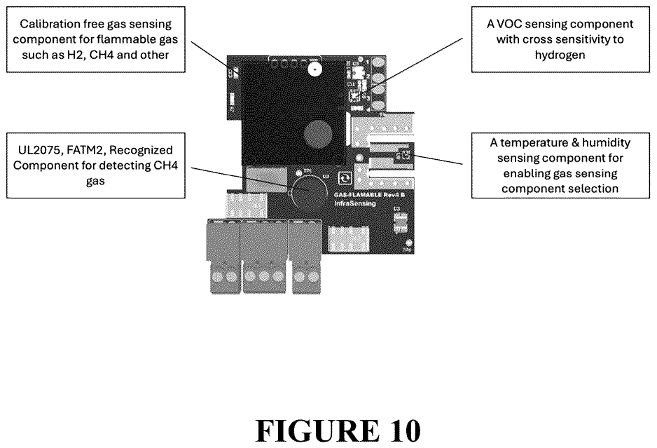

### Definitions

As used herein:

Gas sensing component: a component producing a signal responsive to one or more target gases (for example, but not limited to, electrochemical, catalytic bead, metal oxide semiconductor (MOS), infrared/spectroscopic).

Calibration stable (or “calibration free”) component: a component that provides a stable reference over a prescribed service interval without scheduled manual recalibration.

Agreement criterion: a rule for declaring a validated event, e.g., concurrent threshold exceedance, statistical correlation within a tolerance, classifier based agreement exceeding a confidence level, or a residual error below a limit.

Drifting: a gradual change in a gas sensing component's baseline and/or sensitivity over time due to aging, contamination, poisoning, or environmental exposure, which-if uncorrected-causes measurement error relative to the component's originally calibrated state.

Proxy signal: a signal correlated with concentration of a target gas (e.g., spectral feature subset, multi gas vector, or environmental conditioned surrogate) that is indicative of the target gas and can be used for calibration, drift compensation, cross validation, or interference discrimination when directly measuring the target gas is impractical or ambiguous.

Cross sensitivity: responsiveness to an interfering gas that is not the target gas (e.g., H₂ interfering with CH₄ measurement).

Designated safety standard: any external standard/approval regime imposing detector performance, alarm timing, and fault reporting requirements (e.g., UL 2075, FM), referenced generically herein.

### DETAILED DESCRIPTION OF THE PRESENT INVENTION

In the following detailed description of the present invention of exemplary embodiments of the present invention, reference is made to the accompanying drawings (where like numbers represent like elements), which form a part hereof, and in which is shown by way of illustration specific exemplary embodiments in which the present invention may be practiced. These embodiments are described in sufficient detail to enable those skilled in the art to practice the present invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the present invention. However, it is understood that the present invention may be practiced without these specific details. In other instances, well-known structures and techniques known to one of ordinary skill in the art have not been shown in detail in order not to obscure the present invention. Referring to the figures, it is possible to see the various major elements constituting the apparatus of the present invention.

In one embodiment, a detector 100 includes: a first gas sensing component 110 subject to baseline drift; one or more gas sensing components 120 providing a calibration stable reference and/or corroboration; one or more control units 130 (e.g., processor/microcontroller with memory and I/O); and optional one or more environment sensors 140 (e.g., temperature/humidity/pressure). Components may be mounted on one or more PCBs and enclosed within one or more housings suited to the intended environment. The control unit processes signals from components 110 and 120 to execute automatic calibration, automatic drift compensation, cross validation, adaptive sensing, and cross sensitivity mitigation. Each aspect is independently useful and can be practiced without the others.

The control unit computes a baseline discrepancy between outputs from components 110 and 120. When discrepancy exceeds a tolerance, the control unit adjusts a baseline and/or calibration parameter (e.g., offset, span) of component 110, optionally factoring environmental measurements. Adjustment may occur continuously or periodically, without manual intervention, reducing maintenance and stabilizing long term accuracy. In certification constrained systems, the control unit adjusts the compliance critical component's baseline and/or calibration parameters using outputs from another gas sensing component that serves as a reference source, regardless of whether that reference component is currently recognized under the designated safety standard, without transferring compliance critical signaling to the reference component.

The control unit performs drift compensation by (i) tracking short and long term changes in the first component's zero and span, (ii) confirming suspected drift via one or more calibration stable and/or differently cross sensitive components, and (iii) applying adaptive offset, gain, or transfer function updates in real time or periodically, optionally conditioned on environmental measurements. Drift compensation maintains the recognized component's accuracy over aging and environmental exposure while preserving the recognized component as the sole source of compliance critical signaling. The drift compensation process is applicable whether the reference component is recognized or not under the designated safety standard, ensuring continued operation if the reference component later becomes recognized.

Each component independently detects the same target gas (either directly or via cross sensitivity). The control unit declares a validated event when an agreement criterion between component outputs is satisfied, thereby reducing false alarms and missed events. The agreement criterion may be rule based (threshold, correlation) or model based (e.g., a machine learned classifier) and may require a quorum of N concurrence (e.g., two of three).

The control unit monitors environmental conditions and performance metrics (e.g., stability, range, noise) and environmental or operational conditions including, without limitation, temperature, humidity, pressure, airflow/flow rate, vibration or shock, orientation, and supply voltage, and selects/switches/weights component outputs to form a detection output. For example, above a temperature threshold, the control unit may switch preference from component 110 to component 120; in other conditions, outputs may be blended according to a weighting function.

Where component 110 has a known cross sensitivity to an interferent, component 120 is chosen for a different cross sensitivity profile. The control unit performs differential analysis (e.g., comparing shapes, rates, or steady state values) to suppress, delay, re classify, or adjust a target gas alarm and/or modify a reported concentration when the analysis attributes a response to an interferent, while preserving responsiveness to the target gas. A representative case is methane detection with hydrogen interference.

The detector may be configured for use in systems subject to a designated safety standard. In such cases, compliance critical outputs are generated exclusively from a component designated as the compliance critical path for a designated safety standard, while one or more additional gas sensing components—irrespective of their recognition status—supply calibration, drift compensation, and corroboration data and are prevented by logic and/or hardware from supplanting the compliance critical signaling path. Drift compensation, thresholds, response delays, and fault conditions are parameterized to satisfy the standard without limiting the invention to any one standard.

First Embodiment (Methane, auto calibration): a listed catalytic bead component for CH₄ serves as the primary path, paired with a spectroscopic component providing calibration stable reference; the control unit automatically recalibrates the catalytic bead baseline.

Second Embodiment (H₂, cross validation): an electrochemical component, a spectroscopic component selective to H₂, and a MOS component each respond with differing cross sensitivities; a validated event for a target gas is declared only upon agreement by two or more components according to the agreement criterion.

Third Embodiment (Extreme environment): a catalytic bead component is used in nominal temperature ranges; a MOS component is selected above a threshold temperature.

Fourth Embodiment (Cross sensitivity mitigation): an electrochemical methane detector with Hz cross sensitivity is paired with a component responsive to H₂ but not to CH₄; disagreements prompt modification (including suppression) of methane alarms attributable to H₂.

Electronics may be split across one or more PCBs within one or more enclosures suitable for deployment, with external terminals, indicators, and mounting features as required. The control unit may be implemented via firmware/software on a microcontroller or processor with memory storing instructions for the described algorithms.

The multi technology architecture reduces OPEX, enhances accuracy/reliability, extends service life, and provides redundancy and specificity in safety critical applications.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

What has been described and illustrated herein is a preferred embodiment of the present invention along with some of its variations. The terms, descriptions and figures used herein are set forth by way of illustration only and are not meant as limitations. Those skilled in the art will recognize that many variations are possible within the spirit and scope of the present invention in which all terms are meant in their broadest, reasonable sense unless otherwise indicated. Any headings utilized within the description are for convenience only and have no legal or limiting effect.

Thus, it is appreciated that the optimum dimensional relationships for the parts of the present invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the present invention should be determined by the appended claims and their legal equivalents, rather than by the examples given.

1. A gas detection device, comprising:
a first gas sensing component configured to detect one or more target gases and being subject to baseline drift and/or requiring periodic calibration;
a second gas sensing component configured to detect the same target gas or gases and providing calibration-stable reference data; and
a control unit configured to receive outputs from the first and second gas sensing components, determine a baseline discrepancy of the first gas sensing component based on the second gas sensing component, and automatically adjust a baseline or calibration parameter of the first gas sensing component based on the baseline discrepancy.

2. The device of claim 1, wherein the first and second gas sensing components utilize different sensing technologies.

3. The device of claim 1, wherein the second gas-sensing
component provides a proxy signal correlated with concentration of the target gas, and the control unit uses the proxy signal for calibration, drift compensation, and/or cross-validation.

4. The device of claim 1, wherein the first gas sensing component requires periodic manual calibration and the second gas sensing component is calibration-free.

5. The device of claim 1, wherein the control unit performs continuous or periodic baseline comparison and adjustment.

6. The device of claim 1, wherein the device is configured for use in a system subject to a designated safety standard, and the second gas-sensing component may or may not be recognized under that standard while functioning as a reference source for calibration or drift compensation.

7. The device of claim 6, wherein compliance-critical alarm outputs are generated from the first gas sensing component after said adjustment while the second gas sensing component provides calibration reference.

8. The device of claim 1, wherein compliance-critical detection signaling required by a designated safety standard is generated exclusively from the first gas-sensing component, and the second gas-sensing component is restricted to providing calibration, drift-compensation, and corroboration data and cannot supplant the compliance-critical signaling path.

9. The device of claim 1, wherein the reference component is configured to serve as a calibration and/or drift-compensation source independent of its recognition status under any designated safety standard.

10. The device of claim 1, wherein the first and second gas sensing components are integrated on one or more printed circuit boards within one or more enclosures.

11. A gas detection device, comprising:
at least two gas sensing components utilizing different sensing technologies to detect a same target gas; and a
control unit configured to generate respective detection values from the gas sensing components and to declare a validated gas detection event only when an agreement criterion is satisfied between the detection values.

12. The device of claim 11, wherein the agreement criterion requires both detection values to exceed thresholds, detect similar trends, or exhibit correlation within a tolerance.

13. The device of claim 11, wherein cross-validation reduces false alarms relative to operation of either gas sensing component alone, including reduction of false alarms attributable to cross-sensitivities.

14. The device of claim 11, wherein each gas sensing component operates independently and the control unit provides failover detection upon failure of one gas sensing component.

15. The device of claim 11, wherein cross-validation parameters are configured to satisfy at least one requirement of a designated safety standard.

16. The device of claim 11, wherein the agreement criterion comprises an output of a classifier meeting a confidence threshold.

17. The device of claim 11, wherein a validated event requires concurrence of at least two of three gas-sensing components.

18. A gas detection device, comprising:
at least two gas sensing components targeting a same gas;
at least one environment sensor; and
a control unit configured to select, switch, or weight outputs of the gas sensing components in real time based on an environmental condition to generate a detection output.

19. The device of claim 18, wherein the environmental condition comprises temperature, and the control unit switches to a different gas sensing component when temperature exceeds a threshold.

20. The device of claim 18, wherein
the control unit blends outputs according to a weighting function determined by at least one environmental condition;
selection or weighting is based on at least one of temperature range, humidity range, sensing range, stability, noise, or accuracy; and
environmental or operational conditions comprise one or more of airflow, vibration or mechanical shock, orientation, and supply voltage, and selection or weighting changes when a condition crosses a threshold.
